(19) United States
(12) Patent Application Publication
(10) Pub. No.: US 2025/0031717 A1
(43) Pub. Date: Jan. 30, 2025

(54) METHOD FOR OBTAINING A GELLED PLANT PROTEIN COMPOSITION USING TRANSGLUTAMINASE

(71) Applicants: CORN PRODUCTS DEVELOPMENT, INC., WESTCHESTER, IL (US); KATECH INGREDIENTS SOLUTIONS GMBH, Lübeck (DE)

(72) Inventors: Jocelyn Y HERNANDEZ MEDINA, Hamburg (DE); Alexander MAESSE, Hamburg (DE); Markus TIETJEN, Hamburg (DE)

(21) Appl. No.: 18/704,594

(22) PCT Filed: Oct. 28, 2022
(86) PCT No.: PCT/US2022/048145

§ 371 Date: Apr. 25, 2024

Related U.S. Application Data
(60) Provisional application No. 63/273,202, filed on Oct. 29, 2021.

(51) Int. Cl. A23J 3/14; A23J 1/14; A23J 3/22; C12N 9/10

(52) U.S. Cl. CPC A23J 3/14; A23J 1/148; A23J 3/227; C12N 9/1044; C12Y 203/02013

(57) ABSTRACT

This specification discloses methods for obtaining a gelled plant protein composition using transglutaminase to form gels having high elasticity and gel strength. In one aspect the methods use a highly soluble plant protein or include steps, such as shear. so that at least 40% of the protein relative to the raw material is dissolved in solution prior to adding transglutaminase. The described gelled compositions are useful in alternative meats.

The technology disclosed in this specification pertains to methods for making gelled plant protein compositions using transglutaminase. More specifically, the gelled plant protein compositions are useful as vegan or vegetarian meat alternatives or meat extenders.

Transglutaminase enzymes are used to crosslink various types of proteins. Most relevant to the technologies described in this specification, transglutaminase can be used to link plant proteins to form protein networks capable of forming gelled structures useful as vegan or vegetarian meat alternative, or as meat extenders. For such applications, strong gels are desired, but plant proteins, even from the same source (e.g. pea protein isolates from the same or different vendors) can create gels of variable strength.

This specification provides key variables for ensuring a gelled plant protein composition made using transglutaminase enzyme has high gel strength, which produces firmness, taste, and texture that are particularly desirable for analog meat compositions. This specification also uses the discovered variables to describes methods to make strongly gelled plant protein compositions from various plant protein sources, particularly for use in analog meat compositions. More specifically, obtaining strongly gelled plant protein compositions that are most like animal meat requires that substantial protein is dissolved in solution before adding transglutaminase. Also described in this specification are methods for increasing the dissolved protein content in a solution before adding transglutaminase so that a strongly gelled plant protein composition is obtained.

### BRIEF DESCRIPTION OF THE FIGURES

FIG. 1 depicts weakly gelled plant protein compositions (on the left) compared to a strongly gel plant protein composition (on the right), both being made by methods described in this specification.

FIG. 2 graphs the increase of dissolved protein in water (20° to 30° C.) as shear is applied to a solution of plant protein in water over time.

In one aspect the technology disclosed in this specification pertains to methods of obtaining a solution having high dissolved protein content at relatively low water temperatures. In any embodiment described in this specification a method for obtaining a gelled plant protein composition comprises: providing a plant protein in the form of one or more of a plant protein isolate, plant protein concentrate, or plant flour wherein the plant protein has a solubility in water such that at least about 65% of the plant protein dissolves in in water at 95° C. after centrifuging at 5000 revolutions per minute (rpm) for 20 minutes; mixing the plant protein with water having a temperature of less than about 50° C. or from greater than about 0° C., or from about 10° C., from about 20° C. to about 50° C. or to about 40° C., or to about 30° C. to form a slurry having plant protein content of at least about 3% (wt. % or the slurry) or at least 10%, or at least about 12%; mixing a transglutaminase enzyme with the slurry and incubating the slurry to crosslink at least a portion of the plant protein wherein at least about 40% (wt. % relative to the protein used) at least about 50% of the plant protein is dissolved in water prior to adding transglutaminase, or from about 40%, or from about 50% to about 100% or about 90% or about 80% or two about 70% or to about 60% (wt. % of the total protein).

In at least some embodiments of the methods for making a gelled plant protein composition described in this specification the plant protein is in an amount from about 3%, or from about 4% or from about 5%, or from about 6% or from about 7% or from about 8%, or from about 9%, or from about 10%, or from about 11% or from about 12% to about 50% or to about 40%, or two about 30%, or to about 20%. In at least some embodiments of the described methods the plant protein is in a range selected from the group consisting of from about i) 10% to about 20%, or ii) from about 20% to about 30%, or iii) from about 30% to about 50%.

In any embodiment, described in this specification, a transglutaminase is added to the slurry of plant protein and water in an amount from about 0.001%, or from about 0.01%, or from about 0.05% (wt. % of the slurry), or from about 0.10%, or from about 0.15% or from about 0.20%, or from about 0.30%, or from 0.35%, or from about 0.40% to about 1.00% in a range selected from the group consisting of (i) from about 0.001 to about 0.05%; (ii) from about 0.25% to about 0.35%; (iii) from about 0.27% to about 0.33%; and (iv) from about 0.40 to about 1.00%. In still other embodiments of a method for making a gelled plant protein composition described in this specification, a slurry of plant protein, transglutaminase and water is incubated at a temperature less than about 50° C., or from about 30° C. from about 40° C. to about 50° C. In still other embodiments of a method for making a gelled plant protein composition described in this specification, a slurry of plant protein, transglutaminase and water is incubated for enough time to form a strong gel, or for at least about 30 minutes, or at least about 1 hour or at least about 2 hours or at least about 3 hours.

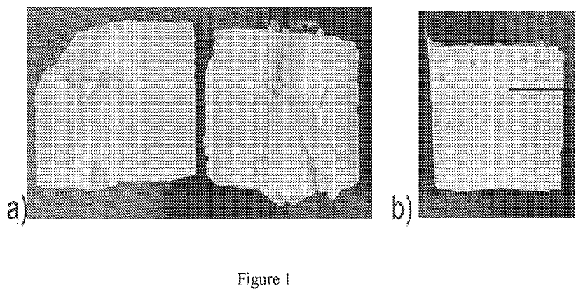

A variety of gel strengths are obtainable using the described methods depending on the protein content in the slurry or amount of protein dissolved in the specification. Within this specification strong gels are characterized as being self-supporting. With reference to FIG. 1, FIG. 1a one the left is a weak gel. The gel visibly separates when a knife is lightly run across the gels surface. FIG. 1b (on the right) is a strong gel that retains its form even after a knife is lightly run across the gel surface. Within this specification, gel strength is also described quantitatively using TA.XT texture analyzer that measures force from a gel on a spherical probe pressed against and through gel composition. Within this specification gel strength was measured under the following conditions: measurements triggered after registering 5 g of force, probe advance maximum depth of 10 mm at a rate of 0.5 mm/sIn this specification, weak gels have a gel strength less than about 100 g. In this specification, moderate gels have a gel strength from about 300 g to about 500 g. In this specification, strong gels have gel strength greater than about 600 g or more preferably 800 g.

For vegan meat alternatives, vegetarian meat alternatives, or meat containing compositions (where the gelled plant protein composition is used to extend or supplement or complement the meat in the composition), commonly, firmer gels are preferred. But it is expected that gel strength will be adjusted to obtain the desired texture. With this context, two observations are made: in this specification a gelled plant protein composition is made in a process that (1) uses at least about 3% (wt. % of the slurry) plant protein provided as a plant flour, plant protein concentrate or plant protein isolate, and that (2) uses soluble protein or process steps so that at least about 40% (wt. % of the protein relative to the starting material) dissolves in water before transglutaminase is added. In any embodiment of the methods described in this specification, weakly gelled plant protein compositions are obtained by mixing from about 3% to about 5% (wt. % of the slurry) plant protein in water. In another embodiment of the methods described in this specification, moderately strongly gelled plant protein compositions are obtained by mixing from about 6% to about 10% (wt. % of the slurry) plant protein. In another embodiment of the methods described in this specification, strongly gelled plant protein compositions are obtained by mixing in an amount greater than about 10% (wt. % of the slurry). With the strongly gelled compositions, particularly for vegan replacement of non-vegan protein sources, the plant protein used content can be adjusted to match the protein of the non-vegan source to be mimicked. For example protein content in the gelled plant protein composition in an amount from about 10% to 15% (wt. % of the composition) approximates the protein content in an egg. As another example protein content in the rage from about 20% to 30% (wt. % of the gelled plant protein composition) approximates the protein content in a meat source.

In any embodiment of the methods described in this specification. to obtain a gelled plant protein composition having a desired gelled strength (weak, moderately strong, strong) at least about 40% (wt. % dissolve protein relative to a plant flour, plant protein concentrate or plant protein isolate) of the plant protein is dissolved in water, or at least about 50% or from about 40%, or about 50% to about 100% or about 90% or about or about 80%, or about 70%, or about 60% before transglutaminase is added.

In some embodiments the methods described in this specification use a plant protein having high solubility in water. In some embodiments, useful plant proteins are plant proteins supplied by a plant flour or a plant protein concentrate, or plant protein isolate wherein at least about 65% (wt. % dissolved protein relative to flour, concentrate, or isolate) of the plant protein dissolves in in water at 95° C., centrifuged at 5000 rpm for 20 minutes. Plant proteins having this degree of solubility include but are not limited to pea protein isolates (about 85% protein (wt. % protein content, dry basis)) available from Ingredion Incorporate, Westchester, Illinois, USA.

Transglutaminase is a heat sensitive enzyme, so in methods described in this specification for making a gelled plant protein composition using high temperature water to dissolve plant protein, for example from about 50° C. to about 99° C., the water is cooled after dissolving protein but before adding transglutaminase. In other embodiments of the methods for making a gelled plant protein composition described in this specification, plant protein is mixed with water having a temperature of less than about 50° C., or in a temperature range about from greater than about 0° C., or from about 10° C., from about 20° C. to about 50° C. or to about 40° C., or to about 30° C., preferably in a range from about 10° C. to about 30° C. In at least some embodiments described in this specification, particularly in embodiments using lower temperatures, the mixture of water and plant protein is mixed using a rotational mixer rotating at a rotational speed from at least about 2000 rpm, or at least about 2500 rpm at least about 3000 rpm or to at most about 5000 rpm or at most about 4000 rpm. In preferred embodiments of the method, the mixing is so that at least about 40% (wt. % dissolved protein relative to the plant flour or plant protein concentrate or plant protein isolate) of the plant protein is dissolved in water or at least about 50%, or from about 40%, or from about 50%, or to about100% or about 90% or about to about 80%, or to about 70%, or to about 60%.

In embodiments where transglutaminase is mixed with water at greater than about 0° C., water may be provided as ice with the shear providing enough energy to the system to melt the ice, which then dissolves at least the plant protein. In at least some embodiments powdered ingredients, at least being plant protein and transglutaminase (in an amounts as described in this specification), are mixed with ice having temperature less than about 0° C. or from about 0° C. to about −10° C. The mixture is mixed with sufficient shear to raise the temperature of the mixture to above 0° C. or from about 1° C. to 10° C. or from about 2° C. and 7° C. In at least some embodiments described in this specification, particularly in embodiments using lower temperatures, the mixture of water and plant protein is mixed using a rotational mixer rotating at a rotational speed from at least about 2000 rpm, or at least about 2500 rpm at least about 3000 rpm or to at most about 5000 rpm or at most about 4000 rpm. In preferred embodiments of the method, the mixing is so that at least about 40% (wt. % dissolved protein relative to the plant flour or plant protein concentrate or plant protein isolate) of the plant protein is dissolved in water or at least about 50%, or from about 40%, or from about 50%, or to about 100% or about 90% or about to about 80%, or to about 70%, or to about 60%.

In any embodiment this specification discloses a method for obtaining a gelled plant protein composition by mixing a plant protein in water having relatively low temperature and using shear so that an intended amount of plant protein is dissolved in the water before adding transglutaminase. In any embodiment, this specification describes a method for obtaining a gelled plant protein composition comprising: mixing a plant protein from one or more of a plant protein isolate, or plant protein concentrate, or plant flour with ice having a temperature less than 0° C., or from about 0° to about −10° C. or with water having a temperature having a temperature of less than about 50° C., or from greater than about 0° C., or from about 10° C., from about 20° C. to about 50° C. or to about 40° C., or to about 30° C., or more preferably from about 10° C., or from about 20° C. to about 30° C. to form a slurry 3% (wt. % or the slurry) or at least 10%, or at least about 12%; applying shear to the slurry so that at least about 40% (wt. % dissolve protein relative in the plant flour, plant protein concentrate or plant protein isolate) of the plant protein is dissolved in water or at least about 50%, or from about 40%, or from about 50% to about to about 80%, or to about 70%, or to about 60%; mixing a transglutaminase enzyme with the slurry in an amount from about 0.001% (wt. % of the slurry) or about 0.01% about 0.05% or from about 0.10%, or from about 0.15%, or from about 0.20% to 0.35%, or to about 0.30% or in a range selected from the group consisting of (i) from about 0.25% to about 0.35%: (ii) from about 0.27% to about 0.33%; and incubating the slurry to crosslink at least a portion of the plant protein. In at least some embodiments of the methods described in this specification, a slurry comprising water, plant protein, and transglutaminase is incubated at a temperature from about 1° C. or from 5° C. or from about 20° C., or from about 30° C., or from about 40° C. to about 50° C., or from about 1° C. to about 10° C., or from about 20°, or from about 30° C., or from about 40° C. to about 50° C.

In at least some embodiments of the methods for making a gelled plant protein composition described in this specification the plant protein is used in an amount from about 3% (wt. % of the mixture), or from about 4% or from about 5%, or from about 6% or from about 7% or from about 8%, or from about 9%, or from about 10%, or from about 11% or from about 12% to about 50% or to about 40%, or two about 30%, or to about 20%. In of the described methods the plant protein is in a range selected from the group consisting of from about i) 10% to about 20% (wt. % of the mixture), or ii) from about 20% to about 30%, or iii) from about 30% to about 50%.

In at least some embodiments of the methods described in this specification, a slurry comprising water, plant protein, and transglutaminase is incubated for a time sufficient to form a strong gel, or for at least about 30 minutes, or at least about 1 hour or at least about 2 hours or at least about 3 hours. In a least some embodiments of the methods described in this specification, a plant protein is mixed with water or aqueous solution having pH from about 5 to 8 to form a slurry.

In at least some embodiments of the methods described in this specification for making a gelled plant protein composition, a transglutaminase enzyme is encapsulated within an encapsulating system to control the release of the transglutaminase into water or an aqueous system. An encapsulated transglutaminase can be used to control release of the transglutaminase enzyme in water and control the occurrence of protein crosslinking. Encapsulating systems may also encapsulate other ingredients to be delivered to the water or aqueous system. For example, in at least some embodiments transglutaminase and plant protein are encapsulated together. In other embodiments, an encapsulating system encapsulates one or more of plant proteins, fibers, and hydrocolloids with transglutaminase. An illustrative encapsulating systems includes one or more films around the transglutaminase, wherein the film breaks down when an environmental parameter is changed, for example the encapsulating system is added to warm water. In at least some embodiments an encapsulating system can break down in water having a temperature from about 30° C., or from about 35°, or from about 40°, or from about 45° to about 50° C. to release at least a transglutaminase enzyme into the water.

In at least some embodiments, an encapsulating system comprises a fat that is solid at temperatures below about 20° C., or from about 35°, or from about 40°, or from about 45° C. In at least some embodiments, an encapsulating system comprise a fat that melts at a temperature above about 20° C. or from about 20° C., or from about 30° C., or from about 35°, or from about 40°, or from about 45° C. In at least some embodiments, the fat encapsulating system would prevent the transglutaminase enzyme from interacting with the substrates below the melting point of the fat. The melting point of the fat is chosen so that it melts during an incubation step as described in this specification so that the transglutaminase enzyme is released into the aqueous phase of a slurry and so that during the incubation the transglutaminase will catalyze a reaction among proteins present in the slurry.

In any embodiment of an encapsulated transglutaminase described in this specification, a useful encapsulating fat a hydrogenated plant oil a coconut oil that has not been hydrogenated or palm oil that has not been hydrogenated or fractionated. Useful hydrogenated or fractionated plants oils include soy oil, sunflower oil, safflower oil, canola oil, rapeseed oil, corn oil, olive oil, and avocado oil. Some embodiments of an encapsulated transglutaminase use hydrogenated coconut oil or a hydrogenated palm oil. In other embodiments of an encapsulated transglutaminase the oil is a blend of more than one oil. In, preferred embodiments, when using a blend of oils, all oils in the blend are plant based. Useful plant-based oils are as previously said in this paragraph include like soy oil, coconut oil, palm oil. sunflower oil, safflower oil, canola oil, rapeseed oil, corn oil, olive oil, and avocado oil, which may be hydrogenated and un-hydrogenated, and fractionated and unfractionated oils

When using a blend of oils, in any embodiment, the blend of oil melts at a temperature above about 20° C. or from about 20° C. or from about 30° C., or from about 35°, or from about 40°, or from about 45° C. When using a blend of oils or blend of plant-based oils, in at least some embodiments at least one of the plant-based oils is an hydrogenated oil or fractionated oil.

In at least some embodiments a transglutaminase enzyme is encapsulated (alone or with a combination of other powdered ingredients) at a temperature less than the inactivation temperature of the transglutaminase enzyme, e.g. less than about 60° C. In at least some embodiments a transglutaminase is encapsulated process mixing at most 40% (wt. %) of a powdered transglutaminase mixture and at least about 60% (wt. %) of fat, wherein the mixing is done with the fat in liquid phase at a temperature below 60° C. and cooling the mixture to a temperature below the melting point of the fat. The cooling process can be any useful process including but not limited to a spray chilling process or use of a fluidizing bed using air or essentially inert gas like nitrogen. Useful transglutaminase mixtures will contain concentrated amounts of transglutaminase relative inert ingredients in the powder so that the transglutaminase has high activity or will comprise a transglutaminase enzyme selected for having high activity. In embodiments transglutaminase will have an activity or at least about 1000 U/g or at least about 1400 U/g, or from about 1000 U/g, or from about 1400 U/g to about 2000 U/g enzymatic activity). The transglutaminase mixture may also include second powdered ingredient, for example a maltodextrin. In any embodiment a transglutaminase mixture comprises transglutaminase and a second powdered ingredient where the transglutaminase is in an amount (wt. %) of at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 75%, or at least about 80%, or at least about 85%, or at least about 90%, or at least about 95%, or at least about 99%, or up to about 100%, wherein the remainder of the transglutaminase mixture comprises a second powdered like maltodextrin, which is used to provide bulk and flow to a powdered transglutaminase composition.

In any embodiment described in this specification the plant protein is mixed with water or ice using a rotational mixer rotating at a rotational speed of at least about at least about 2000 rpm, or at least about 2500 rpm at least about 3000 rpm or to at most about 5000 rpm for a time of at least about 5 minutes, or at least about 7, or at least about 9 than minutes, or from about 5 minutes, or from about 7 minutes, or from about 9 minutes to about 15 minutes, or to about 12 minutes.

In any embodiment described in this specification the plant protein, transglutaminase and ice are mixed using rotational mixer rotating at a rotational speed of at least about at least about 2000 rpm, or at least about 2500 rpm at least about 3000 rpm or to at most about 5000 rpm for a time of at least about 5 minutes, or at least about 7, or at least about 9 than minutes, or from about 5 minutes, or from about 7 minutes, or from about 9 minutes to about 15 minutes, or to about 12 minutes.

In any embodiment of the methods described in this specification, the transglutaminase enzyme is mixed with the slurry of protein using a rotational mixer rotating at a rotation a speed of from about 1000 rpm, or from about 1200 rpm, or from about 1400 rpm to about 2000 rpm, or to about 1800 rpm, or to about 1600 rpm. Transglutaminases are commercially available enzymes. Preferred transglutaminases are from bacterial or non-animal sources, and more preferably it is a non-animal sourced transglutaminase that crosslinks protein in the absence of calcium.

In another embodiment this specification discloses a method for obtaining a gelled plant protein composition comprising: providing a plant protein in the form of one or more of a plant protein isolate, plant protein concentrate, or plant flour, wherein at least about 65% (wt. % dissolved protein relative to the flour, concentrate, or isolate) of the plant protein dissolves in in water at 95° C. after centrifuging at 5000 rpm for 20 minutes mixing the plant protein with liquid water having a temperature of greater than about 50° C., or greater than about 60° C. or greater than about 70° C., or great than about 80° C., or greater than about 90° C., to about 99° C., or to about 95° C. to form a slurry having plant protein content of at least about 3% (wt. % or the slurry), or at least 10%, or at least about 12%; allowing the water to cool to a temperature below about 50° C., from greater than about 0° C., or from about 10° C., from about 20° C. to about 50° C. or to about 40° C., or to about 30° C. mixing a transglutaminase enzyme with the slurry in an amount from 0.001% (wt. % of the slurry) of from about 0.01% or from about 0.05% or from about 0.10%, or from about 0.15% or from about 0.20% to about 0.35% or to about 0.30% or in a range selected from the group consisting of (i) from about 0.25% to about 0.35%: (ii) from about 0.27% to about 0.33%; an shearing the slurry so that at least about 40% (wt. % dissolved protein relative to the plant flour, plant protein concentrate, or plant protein isolate) of the plant protein is dissolved in water or at least about 50%, or from about 40%, or from about 50% to about 100%, or about 90%, or to about 80%, or to about 70%, or to about 60%. The basic method described in this paragraph may be further may be varied using one or more of any aspect of another method for obtaining a gelled plant protein composition described in this specification.

Any plant protein that can be crosslinked using transglutaminase is suitable for the methods described in this specification. In any embodiment of the methods described in this specification the plant protein is a legume protein. In any embodiment of the methods described in this specification the plant protein is selected from the group consisting of pea, fava bean, chickpea, lentil, mung bean, soy and mixtures thereof. Useful non-legume proteins can be sourced from pseudocereals (like amaranth, quinoa, etc), oil seed proteins (like flaxseed, sunflower, rapeseed, etc), nuts (like almond, cashews, etc.), cereals (like rice, wheat, corn, etc.) and mixture thereof. In any embodiment of the methods described in this specification, a plant protein to be crosslinked is high in one or more of lysine and glutamine.

Plant proteins useful in the methods described in this specification may be provided by a plant protein isolate, a plant protein concentrate, a plant flour, or mixtures thereof. In some embodiments the plant protein is provided by a plant flour, which refers to a set of products obtained by milling a plant organ (e.g. tuber, seed (like a nut, legume, cereal etc.)) to obtain a dry powder product having protein content essentially equal to the protein content of the base plant organ. In other embodiments the plant protein is supplied by a plant protein concentration, which refers to a set of dry powder products obtained from a plant source and having a protein content from about 40% to about 70% plant protein by weight (dry basis). Plant protein concentrates are commercially available from various suppliers (for example Ingredion Incorporated) and are derived from various plants (e.g. legumes, nuts, cereals, etc.). In still other embodiments the plant protein is supplied by a plant protein isolate, which refers to a set of dry powder products obtained from a plant source and having plant protein content of greater than about 70% plant protein by weight (dry basis). Plant protein isolates are commercially available from various suppliers (for example Ingredion Incorporated) and are derived from various plants (e.g. legumes, nuts, cereals etc.).

In at least some embodiments of the methods described in this specification the plant protein is provided in the form of a protein isolate having a protein content from about 70% (wt. % dry basis), or about from 75%, or from 80% to about 95%, or to about 90%. Plant protein isolates and legume protein isolates including from pea, fava bean, chickpea, lentil, mung bean, and soy are commercially available from various vendors.

In preferred embodiments, the plant protein is not hydrolyzed by acid or other means to reduce the molecular weight of the proteins used to make the protein slurry. In other preferred embodiments, the plant protein is not deamidated to reduce the number glutamine residues in the protein by converting the glutamine residues to glutamate residues.

The texture or taste of a gelled plant protein composition made by the methods described in this specification can be further modified by adding other ingredients. Useful ingredients that may be used in the processes described in this specification include, but are not limited to, fibers, (including but not limited to cellulosic fibers, non-cellulosic fibers like resistant starch, inulin, and various other short chain fructooligosacchardes), hydrocolloids, fats oils, structure vegetable proteins, salts, seasonings, and flavorings. These ingredients may be added as needed to obtain a desired texture or flavor. The following embodiments describe steps for adding these ingredients individually or blended or added multiply and it is to be understood the steps may be done sequentially in any order or be combined to provide a base formulation comprising one or more or all ingredients said in this paragraph before transglutaminase is added and the formulation is incubated to form the gelled plant protein composition.

In any embodiment described in this specification a method of making a gelled plant protein composition further comprises, after dissolving the protein but before adding transglutaminase, mixing the slurry of plant protein and water with a cellulosic fiber in an amount from about 1% (wt. % of the slurry), or from about 3%, to about 10%, or to about 8%, or to about 7%. In any embodiment the slurry of plant protein, cellulosic fiber. and water is mixed using a rotational mixer rotating at a rotation a speed at least about 2000 rpm, or at least about 2500 rpm at least about 3000 rpm or to at most about 5000 rpm. The shear disperses the fiber in the slurry and may defibrillate or otherwise fragment the fiber to aid in providing texture to the gelled plant protein composition. In any embodiment described in this specification a fiber is any fiber suitable for use in a gelled plant protein composition or an edible gelled plant protein composition.

Illustrative fibers include but are not limited to apple fiber, citrus fiber, pea fiber, potato fiber, wheat fiber, bamboo fiber, carrot fiber, oat fibers (whether soluble), psyllium, and mixtures thereof.

In any embodiment described in this specification a method of making a gelled plant protein composition further comprises, mixing protein, transglutaminase, ice, and a cellulosic fiber in an amount from about 1% (wt. % of the slurry), or from about 3%, to about 10%, or to about 8%, or to about 7%. In any embodiment the of plant protein, transglutaminase, cellulosic fiber, and ice is mixed using a rotational mixer rotating at a rotation a speed at least about 2000 rpm, or at least about 2500 rpm at least about 3000 rpm or to at most about 5000 rpm. The shear melts the ice, dissolves the protein and disperses the fiber to from a slurry. The shear also may defibrillate or otherwise fragment the fiber to aid in providing texture to the gelled plant protein composition. In any embodiment described in this specification a fiber is any fiber suitable for use in a gelled plant protein composition or an edible gelled plant protein composition. Illustrative fibers include but are not limited to apple fiber, citrus fiber, pea fiber, potato fiber, wheat fiber, bamboo fiber, carrot fiber, oat fibers (whether soluble), psyllium, and mixtures thereof.

In any embodiment, this specification discloses a method for making a gelled plant protein composition further comprising mixing a hydrocolloid with the slurry of plant protein and water. \In any embodiment described in this specification wherein, optionally, the hydrocolloid is any hydrocolloid suitable for use in a gelled plant protein composition or an edible gelled plant protein composition. Illustrative hydrocolloids include but are not limited to agar, alginate, carrageenan, cellulose derivates (methylcellulose, carboxymethylcellulose, hydroxypropylmethylcellulose, etc.), guar gum, cassia gum, locust bean gum, konjac gum, konjac mannan, pectin, tara gum, gellan, xanthan, various modified gelling starches, and mixtures.

In any embodiment, this specification discloses a method for making a gelled plant protein composition further comprising adding to the slurry of plant protein and water an oil in an amount greater than 0% (wt. % of the slurry), or at least about 5%, or at least about 10%, or from about 5%, or from about 10%, or from about 15%, to about 30% or about 25% or to about 20%. In any embodiment the slurry of plant protein, oil, and water is mixed to form an emulsion. Useful oils include but are not limited to rapeseed oil, sunflower oil, coconut, and palm oil.

In some embodiments described in this specification at least part of the plant protein in the plant protein slurry is structured plant protein. In some embodiments, the structured plant protein is added to a slurry of plant protein and water after the plant protein has been dissolved in water using the methods described in this specification. In other embodiments, the structure plant protein is added after forming an oil-in-water emulsion or after all high shear steps are completed to avoid damaging the structured plant protein. In still other embodiments, the structured plant protein is hydrated before being added to the slurry of plant protein and water. Structured plant proteins (also called textured vegetable proteins or structured vegetable proteins) are commercially available products commonly made by extruding a base plant protein. Structured plant proteins come in various shapes and sizes, all of which are useful in the methods described in this specification. Any structured plant protein product can be used. Commonly, structured plant proteins are obtained from legume proteins. In any embodiment of the methods described in this specification the structured vegetable protein can be from but is not limited to pea, fava bean, chickpea, lentil, mung bean, soy, and mixtures thereof. In any embodiment, a structure plant protein is added to a slurry or oil in water emulsion to provide protein content to the final gelled plant protein composition in an amount greater than about 20%, or greater than about 25% or greater than about 30% or from about 20% or from about 25% to about 50% or to about 40% or to about 30%.

In any embodiment of a method for making a gelled plant protein composition, following addition of all ingredients, including transglutaminase, the complete mixture is incubated at least about 30 minutes, or at least about 1 hour or at least about 2 hours or at least about 3 hours. The composition may be incubated at a temperature indicated for use of the transglutaminase e.g. less than about 50° C., or from 5° to 50°, or more preferably from 20° and 50° C. For incubation the mixture may be placed in molds where it will gel and take the desired final shape. Generally, at lower temperatures longer incubation periods are needed. In embodiments where protein, transglutaminase, and ice are mixed to form a mixture having temperature of about 0° C. or from about 1° to about 10° C. or from about 2° C. to about 7° C. the mixture may be incubated for more than 10 hours or from 10 hours to 30 hours or from 12 hours to 24 hours.

This specification also discloses a gelled plant protein composition made by any process described in this specification, preferably the composition is a vegan meat alternative or vegetarian meat alternative. The vegan or vegetarian meat alternative may be designed to mimic any meat product and may replace any desired meat source such as red meats (beef, lamb, pork, goat etc.), poultry (chicken, turkey, etc.) seafood (including fish, shellfish, and crustaceans). Using the processes described in this specification the gelled plant protein composition can be formed to mimic processed, cured, or ground meat products. In any embodiment described in this specification, the gelled plant protein product may be incubated in a mold to take the form a patty, a nugget, a strip, a cased sausage, a fish fillet, or a poultry breast.

Following are definitions of select terms used in this specification and other guidance for interpreting the specification.

Within this specification the term “fractionated oil” has its ordinary meaning in the art. Fractionation refers to oil that has been processed to select for fatty acid chain length.

Within this specification the term “plant protein isolate” refers to a set of dry powder products obtained from a plant source and having plant protein content of greater than about 70% plant protein by weight, dry basis. Plant protein isolates are commercially available from various suppliers (for example Ingredion Incorporated) and are derived from various plants (e.g. legumes, nuts, cereals etc.).

Within this specification the term “plant protein concentrate” refers to a set of dry powder products obtained from a plant source and having a protein content from about 40% to about 70% plant protein by weight. Plant protein concentrates are commercially available from various suppliers (for example Ingredion Incorporated) and are derived from various plants types (e.g. legumes, nuts. cereals, etc.).

Within this specification the term “plant flour” refers to a set of products obtained by milling a plant organ (e.g. tuber, seed (like a nut, legume, cereal etc.)) to obtain a dry powder product having protein content essentially equal to the protein content of the base plant organ.

Within this specification, the terms “spray chilling” is used as it is in the prior art. Spray chilling is also known as spray cooling or spray congealing. Without being limited to a particular process, spray chilling involves the addition of the component of interest (e.g. a powdered transglutaminase) to a molten lipid carrier (e.g. a liquid fat) and feeding through an atomizer nozzle that spray the nebulized material is put into an environment that is cooled to below the melting point of the matrix material. The mixture then solidifies and solid lipid microparticles are formed at the same time.

Within this specification the term “transglutaminase” refers to a set of enzymes that primarily catalyze the formation of a bond between y-carboxamide groups of glutamine residue side chains and the e-amino groups of lysine residue side chains to form crosslinks within proteins or between proteins. Transglutaminase enzymes are commercially available and are commonly sold as powdered compositions comprising bulk and flow ingredients such as maltodextrin, which, in this specification, are referred to as transglutaminase.

Within this specification the term “water” is used in the open transitional sense to include liquid water, whether tap water, deionized water, distilled water, etc., and aqueous solutions—i.e. water comprising another dissolved substance. Within this specification water includes the frozen and liquid phases as context dictates.

The abbreviation “rpm” is used throughout this specification and stands for revolutions per minute.

In this specification the abbreviation “U/g” stands for units of activity per gram of transglutaminase, which is used to measure the enzymatic activity of transglutaminase per gram of transglutaminase used. A higher U/g means the transglutaminase can more quickly catalyze the transglutaminase mediated reaction. There are various assays for measuring the transglutaminase activity available commercially. While not exclusively, it is common for the assays to use colorimetry to evaluate the occurrence of the transglutaminase mediated reaction under defined reactions conditions-for example, the linking of a glutamine residue to a lysine reside or of a glutamine reside to a primary amine in solution at temperature around 37° C. and pH 6. These reaction conditions are chosen because they are conditions under which transglutaminase is commonly most active. A colorimetric test measures the change in color of a reaction solution over a defined time (for example 5 minutes) or that was reacted for a defined time (for example 5 minutes) before stopping the reaction. While definitions may differ among assays, in this specification, one “unit” (U) of enzyme catalyzes the formation of 1.0 millimole of crosslink product.

Within this specification the percent (%) of plant protein dissolved in water at 95° C. is determined by the following method. Prepare a 300 ml solution with a protein concentration of 3% (total weight of solution). Mix with a magnetic stirrer until homogenized. Measure the pH. If the pH is below 7 adjust with NaOH (0.1 M). Heat up to 95° C. while stirring. Fill centrifuge tube. If foam was formed during the stirring, avoid pouring the foam when filling the tubes. Tube containing the solution should weigh from about 12.7 g to about 13 g). Place the tube inside a centrifuge and centrifuge the sample (5000 rpm, 20 min). With a pipette, recover 2 ml of supernatant, which is poured onto filter paper above an analyzer. Measure the dry matter of each supernatant (via evaporation of liquid) for example using a OHAUS brand analyzer with a heating element (for example heating solution at 150° C.). Percent of dissolved protein is equal to the dry matter recovered divided by the protein in solution multiplied by 100.

Within this specification “weight percent,” which is abbreviated “wt. %,” when referring to an amount of protein in an isolate, concentrate or flour, unless said otherwise, refers the dry basis weight percentage, meaning the weight percent of protein relative to the total weight of the isolate (etc.) absent the moisture content.

Use of “about” to modify a number is meant to include the number recited plus or minus 10%. Where legally permissible recitation of a value in a claim means about the value. Use of about in a claim or in the specification is not intended to limit the full scope of covered equivalents.

Recitation of the indefinite article “a” or the definite article “the” is meant to mean one or more unless the context clearly dictates otherwise.

While certain embodiments have been illustrated and described, a person with ordinary skill in the art, after reading the foregoing specification, can effect changes, substitutions of equivalents and other types of alterations to the methods, and of the present technology. Each aspect and embodiment described above can also have included or incorporated therewith such variations or aspects as disclosed regarding any or all the other aspects and embodiments.

The present technology is also not to be limited in terms of the aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing aspects only and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

The technology described in this specification can be further understood with reference to the following aspects, which are provided for illustrative purposes and are not intended to limit the full scope of the inventions described in this specification.
1 A method for obtaining a gelled plant protein composition comprising: mixing a plant protein raw material and transglutaminase with water having a temperature of less than about 50° C., or less than about 0° C. from greater than about-10° C., or from about −10° C., or from about 0° C., or from about 10° C., from about 20° C. to about 50° C. to form a mixture having plant protein content of at least about 3% (wt. % or the mixture) or at least 10%, or at least about 12%; applying shear to the mixture so that at least about 40% (wt. % protein relative to the raw material) at least about 50% of the plant protein is dissolved in water prior, or from about 40%, or from about 50 to about 100%, or about 90%, or to about 80% or to about 70% or to about 60% (wt. % dissolved protein relative to the raw material); and incubating the mixture wherein, optionally, the transglutaminase enzyme is encapsulated in an encapsulating system, wherein optionally, the encapsulating system breaks down in water at a temperature from about 20° C., or from about 30°, or from about 35°, or from about 40°, or from about 45° to about 50° C. and wherein, optionally, the mixture is incubated a temperature above the melting temperature of the encapsulating system.
2 The method of claim 1 wherein the water in step has a temperature less than about 0° C. or from about −10° C. to 0° C. and sufficient shear is applied to the mixture so that the mixture has a temperature greater than about 0° C., or from about 1° C. to about 10° C., from about 2° C. to about 7° C.
3 The method of claim 1 or 2 wherein the plant protein raw material is a plant protein isolate, a plant protein concentrate, a plant flour, or mixture thereof.
4 The method of any one of claims 1 to 3 wherein the protein content in the mixture is in an amount from the plant protein is in a or from about 3% (wt. % of the mixture), or from about 4% or from about 5%, or from about 6% or from about 7% or from about 8%, or from about 9%, or from about 10%, or from about 11% or from about 12% to about 50% or to about 40%, or two about 30%, or to about 20%; wherein, optionally, the plant protein is in a range selected from the group consisting of from about i) 10% to about 20% (wt. % of the mixture), or ii) from about 20% to about 30%, or iii) from about 30% to about 50%.
5 The method of any one of claims 1 to 4 wherein the transglutaminase is mixed with the slurry in an amount from about 0.001% (wt. % of the slurry) or from about 0.01%, or from about 0.05%, or from about 0.10%, or from about 0.15%, or from about 0.20%, or from about 0.30%, or from about 0.35%, or from about 0.40%, or to about 1.00%, wherein, preferably, the transglutaminase is in an amount from about 0.40% to about 1.00%.
6 The method of any one of claims 1 to 5 wherein the mixture is incubated for at least about 30 minutes, or at least about 1 hour or at least about 2 hours or at least about 3 hours, or at least about 10 hours, or from about hour to about 30 hours or from about 12 hours to about 24 hours.
7 The method of any one of claims 1 to 6 wherein the mixture is incubated a temperature greater than about 0° C., or from about 1° C. to about 10° C., from about 2° C. to about 7° C. for a at least about 10 hours or from about 10 to about 30 hours, or from about 12 to about 24 hours.
8 The method of any one of claims 1 to 7 wherein the water has pH from about 5 to about 8.
9 The method of any one of claims 1 to 8 wherein the water has temperature from about 10° C. or from about 20° C. to about 30° C.
10 The method of any one of claims 1 to 9 wherein the shear is applied by mixing the slurry of plant protein and water using a rotational mixer rotating at a rotational speed at least about 2000 rpm, or at least about 2500 rpm at least about 3000 rpm or to at most about 5000 rpm.
11 The method of any one of claims 1 to 10 wherein the mixture of step a) is mixed at a temperature less than about 0° C. or from about −10° C. to 0° C. and wherein the shear is applied to the mixture using a rotational mixer rotating at a rotation speed at least about 2000 rpm, or at least about 2500 rpm at least about 3000 rpm or to at most about 5000 rpm.
12 The method of any one of claims 1 to 11 wherein the shear is applied to the mixture using a rotational mixer rotating at a rotational speed of at least about 2000 rpm, or at least about 2500 rpm at least about 3000 rpm or to at most about 5000 rpm for a time of at least about 5 minutes, or at least about 7, or at least about 9, or from about 5 minutes, or from about 7 minutes, or from about 9 minutes to about 15 minutes, or to about 12 minutes.
13 The method of any one of claims 1 to 12 wherein the plant protein is a legume protein, wherein optionally the plant protein is selected from the group consisting of pea, fava bean, chickpea, lentil, mung bean, and soy mixtures thereof.
14 The method of any one of claims 1 to 13 further comprising in step a) mixing a cellulosic fiber in an amount from about 1% (wt. % of the slurry) or from about 3%, to about 10%, or to about 8%, or to about 7% wherein, optionally, the fiber is selected from the group consisting of apple fiber, citrus fiber, pea fiber, potato fiber, wheat fiber, bamboo fiber, carrot fiber, psyllium, and mixtures thereof.
15 The method of any one of claims 1 to 14 further comprising in step a) mixing a hydrocolloid wherein, optionally, the hydrocolloid is selected from the group consisting of agar, alginate, carragenan, methylcellulose, carboxymethylcellulose, hydroxypropylmethylcellulose, guar gum, cassia gum, locust bean gum, konjac gum, konjac mannan, pectin, tara gum, gellan, xanthan, various modified gelling starches, and mixtures thereof.
16 The method of any one of claims 1 to 15 comprising mixing in step a) an oil in an amount of at least about 5%, or at least about 10%, or from about 5% (wt. % of the mixture), or from about 10%, or from 15%, to about 30%, or to about 25%, or to about 20% wherein the mixing forms an emulsion.
17 The method of any one of claims 1 to 16 wherein the plant protein is a plant protein isolate having a protein content from about 70% (wt. % protein content, dry basis), or about from 75%, or from 80% to about 95%, or to about 90.
18 The method of any one of claims 1 to 17 wherein the plant protein is not deamidated or hydrolyzed.
19 The method of any one of claims 1 to 18 further comprising in step a) mixing a structured plant protein wherein optionally the structured plant protein is a legume protein, wherein optionally the plant protein is selected from the group consisting of pea, fava bean, chickpea, lentil and mixtures thereof.
20 The method of any one of claims 1 to 19 further comprising placing at least part of the slurry comprising transglutaminase in a mold and allowing the slurry to incubate.
21 An encapsulated transglutaminase comprising: transglutaminase in an amount from about 1% to about to about 40%, or from about 5% to about 40%, or from about 10% to about 40%, or from about 15% to about 40%, or from about 20% to about 40%, or from about 25% to about 40%, or from about 30% to about 40%, or from about 35% to about 40% (wt. %); and an oil in an amount from about 60% to about 99% or from about 60% to about 95%, or from about 60% to about 90%, or from about 60% to about 85%, or from 60% to about 80%, or from about 60% to about 75%, or from 60% to about 70%, or from about 60%, to about 65% (wt. %) wherein the oil has a melting point from about 20° C., or from about 30°, or from about 35°, or from about 40°, or from 45° to about 50° C. wherein preferably the oil is an amount from about 70% to about 60% or from about 65% to about 60%.
22 The encapsulated transglutaminase of any one of claims 21 wherein the transglutaminase in an amount from about from about 30% to about 40%, or from about 35% to about 40% (wt. %); and the oil is in an amount from about 60% to about 99% or from about 60% to about 95%, or from about 60% to about 90%, or from about 60% to about 85%, or from 60% to about 80%, or from about 60% to about 75%, or from 60% to about 70%, or from about 60%, to about 65% (wt. %).
23 The encapsulated transglutaminase of claim 21 wherein the transglutaminase in an amount from about from about 15% to about 25% (wt. %); and the oil is in an amount from about 60% to about 99% or from about 60% to about 95%, or from about 60% to about 90%, or from about 60% to about 85%, or from 60% to about 80%, or from about 60% to about 75%, or from 60% to about 70%, or from about 60%, to about 65% (wt. %)
24 The encapsulated transglutaminase of claim 21 wherein the transglutaminase in an amount from about from about 5 to 10% (wt. %); and an oil in an amount from about 60% to about 99% or from about 60% to about 95%, or from about 60% to about 90%, or from about 60% to about 85%, or from 60% to about 80%, or from about 60% to about 75%, or from 60% to about 70%, or from about 60%, to about 65% (wt. %)
25 The encapsulated transglutaminase of claims 21 to 24 wherein the oil is a hydrogenated plant oil a fractionated plant oil, a coconut oil that has not been hydrogenated o fractionated, or palm oil that has not been hydrogenated or fractionated.
26 The encapsulated transglutaminase of claims 21 to 25 wherein the is a hydrogenated plant oil or fractionated plant oil that is selected from the group consisting of soy oil, coconut oil, palm oil, sunflower oil, safflower oil, canola oil, rapeseed oil, corn oil, olive oil, and avocado oil.
27 The encapsulated transglutaminase of any one of claims 21 to 26 wherein the oil is a hydrogenated or fractionated coconut oil or a hydrogenated or fractionated palm oil.
28 The encapsulated transglutaminase of any one of claims 21 to 27 wherein the oil is a blend of more than one oil.
29 The encapsulated transglutaminase of claim any one of claims 21 to 28 wherein the oil is a blend of more than one oil and at least one of the oils is selected from the groups consisting of soy oil, coconut oil, palm oil, sunflower oil, safflower oil, canola oil, rapeseed oil, corn oil, olive oil, avocado oil, and mixtures thereof; optionally, wherein, all the oils in are plant oils.
30 The encapsulated transglutaminase of any one of claims 21 to 29 wherein the oil is a blend of more than one oil wherein at least one of the oils in the blend is an hydrogenate oil; wherein, optionally, the hydrogenated oil is selected from the group consisting of soy oil, coconut oil, palm oil, sunflower oil, safflower oil, canola oil, rapeseed oil, corn oil, olive oil, avocado oil, and mixtures thereof, optionally, wherein, all the oils in are plant oils.
31 The encapsulated transglutaminase of any one of claims 21 to 30 further comprising a maltodextrin wherein the maltodextrin is in amount from about 1% to about to about 40%, or from about 5% to about 40%, or from about 10% to about 40%, or from about 15% to about 40%, or from about 20% to about 40%, or from about 25% to about 40%, or from about 30% to about 40%, or from about 35% to about 40% (wt. %).
32 The encapsulated composition of any one of claims 21 to 31 wherein the transglutaminase and maltodextrin are in a ratio (transglutaminase to maltodextrin) within a range of from 9:1, or from 8:1, or from 7:1 or from 6:1, or from 5:1 m or from 3:1, or 2:1 to 1:1.
33 The encapsulated composition of any one of claims 21 to 32 wherein the transglutaminase and maltodextrin are in a ratio (transglutaminase to maltodextrin) within a range from 1:1 to 1:2, or to 1:3, or to 1:4, or to 1:5, or to 1:6, or to 1:7, or to 1:8, or to 1:9.
34 The encapsulated transglutaminase of any one of claims 21 to 33 wherein the transglutaminase has an enzyme activity of at least 1000 U/g or from about 1000 U/g, or from about 1400 U/g to about 2000 U/g enzymatic activity.
35 A method for encapsulating transglutaminase comprising: mixing at a temperature from 30° to 60° C. a liquid oil having a melting point melting point from about 20° C., or from about 30°, or from about 35°, or from about 40°, or from 45°, to about 50° C. with a transglutaminase mixture comprising a transglutaminase enzyme to form an oil mixture; and cooling the mixture to a temperature below the melting temperature of the oil wherein the transglutaminase mixture is in an amount in an amount from about 1% to about to about 40%, or from about 5% to about 40%, or from about 10% to about 40%, or from about 15% to about 40%, or from about 20% to about 40%, or from about 25% to about 40%, or from about 30% to about 40%, or from about 35% to about 40% (wt. %) of the oil mixture; and wherein, the oil is in an amount from about 60% to about 99% or from about 60% to about 95%, or from about 60% to about 90%, or from about 60% to about 85%, or from 60% to about 80%, or from about 60% to about 75%, or from 60% to about 70%, or from about 60%, to about 65% (wt. %) of the oil mixture wherein, preferably, the oil is an amount from about 70% to about 60% or from about 65% to about 60% (wt. %) of the oil mixture; and wherein, preferably, the transglutaminase mixture is in an amount from about 30%, from about 35% to about 40% (wt. %) of the oil mixture.
36 The method of claim 35 wherein the transglutaminase is in an amount from about from about 30% to about 40%, or from about 35% to about 40% (wt. %) of the transglutaminase mixture; and the oil is in an amount from about 60% to about 99% or from about 60% to about 95%, or from about 60% to about 90%, or from about 60% to about 85%, or from 60% to about 80%, or from about 60% to about 75%, or from 60% to about 70%, or from about 60%, to about 65% (wt. %) of the oil mixture.
37 The method of claim 35 wherein the transglutaminase in an amount from about from about 15% to about 25% (wt. %) of the transglutaminase mixture; and the oil is in an amount from about 60% to about 99% or from about 60% to about 95%, or from about 60% to about 90%, or from about 60% to about 85%, or from 60% to about 80%, or from about 60% to about 75%, or from 60% to about 70%, or from about 60%, to about 65% (wt. %) of the oil mixture.
38 The method of claim 35 wherein the transglutaminase in an amount from about from about 5 to 10% (wt. %) of the transglutaminase mixture; and an oil in an amount from about 60% to about 99% or from about 60% to about 95%, or from about 60% to about 90%, or from about 60% to about 85%, or from 60% to about 80%, or from about 60% to about 75%, or from 60% to about 70%, or from about 60%, to about 65% (wt. %) of the oil mixture.
39 The method of any one of claims 35 to 38 wherein the oil is a hydrogenated plant oil or a coconut oil or palm oil that has not been hydrogenated.
40 The method of any one of claims 35 to 39 wherein the oil is a hydrogenated coconut oil or a hydrogenated palm oil.
41 The method of any one of claims 35 to 40 wherein the hydrogenated plant oil is selected from the groups consisting of soy oil, coconut oil, palm oil, sunflower oil, safflower oil, canola oil, rapeseed oil, corn oil, olive oil, and avocado oil.
42 The method of any one of claims 35 to 41 wherein the oil is a blend of more than one oil.
43 The method of any one of claims 35 to 42 wherein the oil is a blend of more than one oil and at least one of the oils is selected from the groups consisting of soy oil, coconut oil, palm oil, sunflower oil, safflower oil, canola oil, rapeseed oil, corn oil, olive oil, avocado oil and mixtures thereof.
44 The method of any one of claims 35 to 43 wherein the oil is a blend of more than one oil wherein at least one of the oils in the blend is an hydrogenate oil.
45 The method of any one of claims 35 to 44 wherein the transglutaminase mixture further comprises a maltodextrin in amount from about 1% to about to about 40%, or from about 5% to about 40%, or from about 10% to about 40%, or from about 15% to about 40%, or from about 20% to about 40%, or from about 25% to about 40%, or from about 30% to about 40%, or from about 35% to about 40% (wt. %).
46 The method of any one of claims 35 to 45 wherein, in the transglutaminase mixture, the transglutaminase and maltodextrin are in a ratio (transglutaminase to maltodextrin) within a range of from 9:1, or from 8:1, or from 7:1 or from 6:1, or from 5:1 m or from 3:1, or 2:1 to 1:1.
47 The method of any one of claims 35 to 46 wherein, in the transglutaminase mixture, the transglutaminase and maltodextrin are in a ratio (transglutaminase to maltodextrin) within a range from 1:1 to 1:2, or to 1:3, or to 1:4, or to 1:5, or to 1:6, or to 1:7, or to 1:8, or to 1:9.
48 The method of any one of claims 71 to 83 wherein the transglutaminase has an enzyme activity of at least about 1000 U/g or from about 1000 U/g or from about 1400 U/g to about 2000 U/g enzymatic activity.
49 A method for obtaining a gelled plant protein composition comprising: providing a plant protein in the form of one or more of a plant protein isolate, plant protein concentrate, or plant flour wherein the plant protein has a solubility such that at least about 65% (wt. % dissolved protein relative to the flour, concentrate, or isolate) of the plant protein dissolves in water at 95° C. after centrifuging at 5000 rpm for 20 minutes; mixing the plant protein with water having a temperature of less than about 50° C., or from greater than about 0° C., or from about 10° C., from about 20° C. to about 50° C. or to about 40° C., or to about 30° C. to form a slurry having plant protein content of at least about 3% (wt. % of the slurry) or at least 10%, or at least about 12%; mixing a transglutaminase enzyme; and incubating the slurry wherein at least about 40% (wt. % of dissolved protein relative to plant flour, or plant protein concentrate, or plant protein isolate) at least about 50% of the plant protein is dissolved in water prior to adding transglutaminase, or from about 40%, or from about 50% to about 100%, or about 90%, or to about 80%, or to about 70%, or to about 60%; wherein, optionally, the transglutaminase enzyme is encapsulated in an encapsulating system, wherein optionally, the encapsulating system breaks down in water at a temperature from about 30°, or from about 35°, or from about 40°, or from about 45° to about 50° C.
50 The method of claim 49 wherein the protein content in the slurry is in an amount from the plant protein is in a or from about 3% (wt. % of the slurry), or from about 4% or from about 5%, or from about 6% or from about 7% or from about 8%, or from about 9%, or from about 10%, or from about 11% or from about 12% to about 50% or to about 40%, or two about 30%, or to about 20%; wherein, optionally, the plant protein is in a range selected from the group consisting of from about i) 10% to about 20%, or ii) from about 20% to about 30%, or iii) from about 30% to about 50% (wt. % of the slurry).
51 The method of claim 49 or 50 wherein transglutaminase is added to the slurry of plant protein and water in an amount from about 0.001% (wt. % of the slurry) or from about 0.01%, or from about 0.05%, or from about 0.10%, or from about 0.15% or from about 0.20% to about 0.35% or to about 0.30% or in a range selected from the group consisting of (i) from about 0.001% to about 0.05% (wt. % of the slurry); (ii) from about 0.25% to about 0.35%; and (iii) from about 0.27% to about 0.33%.
52 The method of any one of claims 40 to 51 wherein the mixing uses a rotational mixer rotating at least about 2000 rpm, or at least about 2500 rpm at least about 3000 rpm or to at most about 5000 rpm; wherein, optionally, the mixing applies shear to the slurry so that at least about 40% (wt. % of dissolved protein relative to plant flour, or plant protein concentrate, or plant protein isolate) at least about 50% of the plant protein is dissolved in water, or at least about 50%, or from about 40%, or about 50% to about 100%, or about 90%, or to about 80% or to about 70% or to about 60%.
53 The method of any one of claims 49 to 52 wherein the slurry is incubated for at least about 30 minutes, or at least about 1 hour or at least about 2 hours or at least about 3 hours.
54 The method of any one of claims 49 to 53 wherein the plant protein is mixed with water at a temperature of from about 10° C., or from about 20° to about 30° C. wherein, optionally the mixing uses a rotational mixer rotating at a rotational speed at least about 2000 rpm, or at least about 2500 rpm at least about 3000 rpm or to at most about 5000 rpm; wherein, optionally, the mixing applies shear to the slurry so that at least about 40% (wt. % of dissolved protein relative to plant flour, or plant protein concentrate, or plant protein isolate) of the plant protein is dissolved in water, or at least about 50%, or from about 40%, or about 50% to about 100%, or about 90%, or to about 80% or to about 70% or to about 60%.
55 The method of any one of claims 49 to 54 wherein the plant protein is mixed with water using a rotational mixer rotating at a rotational speed at least about 2000 rpm, or at least about 2500 rpm at least about 3000 rpm or to at most about 5000 rpm for a time of at least about 5 minutes, or at least about 7, or at least about 9 minutes, or from about 5 minutes, or from about 7 minutes, or from about 9 minutes to about 15 minutes, or to about 12 minutes.
56 The method of any one of claims 49 to 55 wherein the transglutaminase enzyme is mixed with the slurry using a rotational mixer rotating at a rotation a speed of from about 1000 rpm, or from about 1200 rpm, or from about 1400 rpm to about 2000 rpm, or to about 1800 rpm, or to about 1600 rpm.
57 The method of any one of claims 49 to 56 wherein the plant protein is a legume protein, wherein optionally the plant protein is selected from the group consisting of pea, fava bean, chickpea, lentil, mung bean and soy and mixtures thereof.
58 The method of any one of claims 49 to 57 further comprising, between steps b) and c) mixing the slurry of plant protein and water with a cellulosic fiber in an amount from about 1% (wt. % of the mixture), or from about 3%, to about 10%, or to about 8%, or to about 7% wherein, optionally, the mixing of the cellulosic fiber with the slurry of plant protein, water uses a rotational mixer rotating at a rotation a speed at least about 2000 rpm, or at least about 2500 rpm at least about 3000 rpm or to at most about 5000 rpm; wherein, optionally, the fiber is selected from the group consisting of apple fiber, citrus fiber, pea fiber, potato fiber, wheat fiber, bamboo fiber, carrot fiber, psyllium, and mixtures thereof.
59 The method of any one of claims 49 to 58 further comprising mixing a hydrocolloid with the slurry of plant protein and water wherein, optionally, the hydrocolloid is selected from the group consisting of agar, alginate, carrageenan, methylcellulose, carboxymethylcellulose, hydroxypropylmethylcellulose, guar gum, cassia gum, locust bean gum, konjac gum, konjac mannan, pectin, tara gum, gellan, xanthan, various modified gelling starches, and mixtures.
60 The method of any one of claims 49 to 59 further comprising adding to the slurry of plant protein and water an oil in an amount at greater than 0%, or least about 5%, or at least about 10%, or from about 5% (wt. % of the slurry), or from about 10%, or from about 15%, to about 30% or about 25% or to about 20% wherein the slurry of plant protein, oil and water is mixed to form an emulsion.
61 The method of any one of claims 49 to 60 wherein the plant protein is a plant protein isolate having a protein content from about 70% (wt. % protein content (dry basis), or about from 75%, or from 80% to about 95%, or to about 90%.
62 The method of any one of claims 49 to 61 wherein the plant protein is not deamidated or hydrolyzed.
63 The method of any one of claims 49 to 62 wherein at least part of the plant protein is a structured plant protein wherein, optionally, the structured plant protein is obtained from a legume protein, wherein, optionally, the plant protein is selected from the group consisting of pea, fava bean, chickpea, lentil, mung bean, and soy and mixtures thereof.
64 The method of any one of claims 49 to 63 further comprising mixing a structured plant protein with a slurry of plant protein and water or an oil in water emulsion wherein, optionally, the structured plant protein is obtained from a legume protein, wherein, optionally, the plant protein is selected from the group consisting of pea, fava bean, chickpea, lentil, mung bean, and soy and mixtures thereof.
65 The method of any one of claims 49 to 64 further comprising placing at least part of the slurry comprising transglutaminase in a mold and allowing the slurry to incubate.
66 A method for obtaining a gelled plant protein composition comprising: mixing the plant protein raw material, with water having a temperature of less than about 50° C., or from greater than about 0° C., or from about 10° C., from about 20° C. to about 50° C. or to about 40° C., or to about 30° C. to form a slurry having plant protein content of at least about 3% (wt. % or the slurry) or at least 10%, or at least about 12%; applying shear to the slurry so that at least about 40% (wt. % protein relative to the raw material) at least about 50% of the plant protein is dissolved in water prior to adding transglutaminase, or from about 40%, or from about 50 to about 100%, or about 90%, or to about 80% or to about 70% or to about 60% (wt. % dissolved protein relative to the raw material); mixing a transglutaminase enzyme with the slurry; and incubating the slurry wherein, optionally, the transglutaminase enzyme is encapsulated in an encapsulating system, wherein optionally, the encapsulating system breaks down in water at a temperature from about 30°, or from about 35°, or from about 40°, or from about 45° to about 50° C.
67 The method of claim 66 wherein the plant protein raw material is a plant protein isolate, a plant protein concentrate, a plant flour, or mixture thereof.
68 The method of claim 66 or 67 wherein the protein content in the slurry is in an amount from the plant protein is in a or from about 3% (wt. % of the slurry), or from about 4% or from about 5%, or from about 6% or from about 7% or from about 8%, or from about 9%, or from about 10%, or from about 11% or from about 12% to about 50% or to about 40%, or two about 30%, or to about 20%; wherein, optionally, the plant protein is in a range selected from the group consisting of from about i) 10% to about 20% (wt. % of the slurry), or ii) from about 20% to about 30%, or iii) from about 30% to about 50%.
69 The method of any one of claims 66 to 68 wherein the transglutaminase is mixed with the slurry in an amount from about 0.001% (wt. % of the slurry) or from about 0.01%, or from about 0.05%, or from about 0.10%, or from about 0.15%, or from about 0.20% to 0.35%, or to about 0.30%, or in a range selected from the group consisting of (i) from about 0.001% to about 0.05%; (ii) from about 0.25% to about 0.35%; and (iii) from about 0.27% to about 0.33%.
70 The method of any one of claims 66 to 69 wherein the slurry is incubated for at least about 30 minutes, or at least about 1 hour or at least about 2 hours or at least about 3 hours.
71 The method of any one of claims 66 to 70 wherein the water has pH from about 5 to about 8.
72 The method of any one of claims 66 to 71 wherein the water has temperature from about 10° C. or from about 20° C. to about 30° C.
73 The method of any one of claims 66 to 72 wherein the shear is applied by mixing the slurry of plant protein and water using a rotational mixer rotating at a rotational speed at least about 2000 rpm, or at least about 2500 rpm at least about 3000 rpm or to at most about 5000 rpm.
74 The method of any one of claims 66 to 73 wherein the plant protein is mixed with water at a temperature of from about 20° to about 30° C. wherein, optionally the mixing uses a rotational mixer rotating at a rotation speed at least about 2000 rpm, or at least about 2500 rpm at least about 3000 rpm or to at most about 5000 rpm; wherein, optionally, the mixing applies shear to the slurry so that at least about 40% (wt. % dissolved protein relative to the raw material) of the plant protein is dissolved in water, or at least about 50%, or from about 40%, or about 50% to about 100%, or about 90%, or to about 80% or to about 70% or to about 60%.
75 The method of any one of claims 66 to 74 wherein the plant protein is mixed with water using a rotational mixer rotating at a rotational speed of at least about 2000 rpm, or at least about 2500 rpm at least about 3000 rpm or to at most about 5000 rpm for a time of at least about 5 minutes, or at least about 7. or at least about 9, or from about 5 minutes, or from about 7 minutes, or from about 9 minutes to about 15 minutes, or to about 12 minutes.
76 The method of any one of claims 66 to 75 wherein the transglutaminase enzyme is mixed with the slurry using a rotational mixer rotating at a rotation a speed of from about 1000 rpm, or from about 1200 rpm, or from about 1400 rpm to about 2000 rpm, or to about 1800 rpm, or to about 1600 rpm.
77 The method of any one of claims 66 to 76 wherein the plant protein is a legume protein, wherein optionally the plant protein is selected from the group consisting of pea, fava bean, chickpea, lentil, mung bean, and soy mixtures thereof.
78 The method of any one of claims 66 to 77 further comprising mixing with the slurry of plant protein and water a cellulosic fiber in an amount from about 1% (wt. % of the slurry) or from about 3%, to about 10%, or to about 8%, or to about 7% wherein, optionally, the fiber is mixed with the slurry of plant protein and fiber using a rotational mixer rotating at a rotation a speed of at least about 2000 rpm, or at least about 2500 rpm at least about 3000 rpm or to at most about 5000 rpm; wherein, optionally, the fiber is selected from the group consisting of apple fiber, citrus fiber, pea fiber, potato fiber, wheat fiber, bamboo fiber, carrot fiber, psyllium and mixtures thereof.
79 The method of any one of claims 66 to 78 further comprising mixing a hydrocolloid with the slurry of plant protein and water wherein, optionally, the hydrocolloid is selected from the group consisting of agar, alginate, carragenan, methylcellulose, carboxymethylcellulose, hydroxypropylmethylcellulose, guar gum, cassia gum, locust bean gum, konjac gum, konjac mannan, pectin, tara gum, gellan, xanthan, various modified gelling starches, and mixtures thereof.
80 The method of any one of claims 66 to 79 further comprising adding to the slurry of plant protein and water an oil in an amount greater than 0% (wt. % of the slurry), or at least about 5%, or at least about 10%, or from about 5% (wt. % of the slurry), or from about 10%, or from 15%, to about 30%, or to about 25%, or to about 20% wherein the slurry of plant protein, oil and water is mixed to form an emulsion.
81 The method of any one of claims 66 to 80 wherein the plant protein is a plant protein isolate having a protein content from about 70% (wt. % protein content, dry basis), or about from 75%, or from 80% to about 95%, or to about 90.
82 The method of any one of claims 66 to 81 wherein the plant protein is not deamidated or hydrolyzed.
83 The method of any one of claims 66 to 82 wherein at least part of the plant protein is a structured protein wherein, optionally, the structured plant protein is a legume protein, wherein optionally the plant protein is selected from the group consisting of pea, fava bean, chickpea, lentil, mung bean, and soy and mixtures thereof.
84 The method of any one of claims 66 to 83 further comprising mixing a structured plant protein to a slurry of plant protein and water wherein optionally the structured plant protein is a legume protein, wherein optionally the plant protein is selected from the group consisting of pea, fava bean, chickpea, lentil, and mixtures thereof.
85 The method of any one of claims 66 to 84 further comprising placing at least part of the slurry comprising transglutaminase in a mold and allowing the slurry to incubate.
86 A method for obtaining a gelled plant protein composition comprising: providing a plant protein raw material wherein at least about 65% (wt. % dissolved protein relative to the raw material) of the plant protein dissolves in in water at 95° C. after centrifuging at 5000 rpm for 20 minutes; mixing the plant protein with liquid water having a temperature of greater than about 50° C., or greater than about 60° C. or greater than about 70° C., or great than about 80° C., or greater than about 90° C., to about 99° C., or to about 95° C. to form a slurry having plant protein content of at least about 3% (wt. % or the slurry) or at least 10%, or at least about 12%; allowing the water to cool to a temperature below about 50° C., from greater than about 0° C., or from about 10° C., from about 20° C. to about 50° C. or to about 40° C., or to about 30° C.; mixing a transglutaminase enzyme with the slurry; and incubating the slurry wherein, optionally, the transglutaminase enzyme is encapsulated in an encapsulating system, wherein optionally, the encapsulating system breaks down in water at a temperature from about 30°, or from about 35°, or from about 40°, or from about 45° to about 50° C.
87 The method of claim 86 wherein the plant protein raw material is supplied in the form of a plant protein isolate, a plant protein concentrate, a plant flour, or mixtures thereof.
88 The method of claim 86 or 87 wherein at least about 40% (wt. % dissolved protein relative to the raw material) at least about 50% of the plant protein is dissolved in water prior to adding transglutaminase, or from about 40%, or from about 50% or about 100%, or to about 90%, or to about 80% or two about 70% or to about 60%.
89 The method of any one of claims 86 to 88 wherein the protein content in the slurry is in an amount from the plant protein is in a or from about 3% (wt. % of the slurry), or from about 4% or from about 5%, or from about 6% or from about 7% or from about 8%, or from about 9%, or from about 10%, or from about 11% or from about 12% to about 50% or to about 40%, or two about 30%, or to about 20%; wherein, optionally, the plant protein is in a range selected from the group consisting of from about i) 10% to about 20% (wt. % of the slurry), or ii) from about 20% to about 30%, or iii) from about 30% to about 50%.
90 The method of any one of claims 86 to 89 wherein the transglutaminase is mixed with the slurry in an amount from about 0.001% (wt. % of the slurry) or from about 0.01%, or from about 0.05% or from about 0.10%, or from about 0.15% or from about 0.20% to about 0.35% or to about 0.30% or in a range selected from the group consisting of (i) from about 0.001% (wt. % of the slurry) to about 0.05%; (ii) from about 0.25% to about 0.35%; and (iii) from about 0.27% to about 0.33%.
91 The method of any one of claims 86 to 90 wherein the slurry is incubated for at least about 30 minutes, or at least about 1 hour or at least about 2 hours or at least about 3 hours.
92 The method of any one of claims 86 to 91 wherein the plant protein is mixed with water using a rotational mixer rotating at a rotational speed of at least about 2000 rpm, or at least about 2500 rpm at least about 3000 rpm or to at most about 5000 rpm for a time of at least about 5 minutes, or at least about 7, or at least about 9 minutes, or from about 5 minutes, or from about 7 minutes, or from about 9 minutes to about 15 minutes, or to about 12 minutes.
93 The method of any one of claims 86 to 92 wherein the transglutaminase enzyme is mixed with the slurry using a rotational mixer rotating at a rotation a speed of from about 1000 rpm, or from about 1200 rpm, or from about 1400 rpm to about 2000 rpm, or to about 1800 rpm, or to about 1600 rpm.
94 The method of any one of claims 86 to 93 wherein the plant protein is a legume protein, wherein optionally the plant protein is selected from the group consisting of pea, fava bean, chickpea, lentil, mung bean and soy and mixtures thereof.
95 The method of any one of claims 86 to 94 further comprising, between steps b) and c) mixing the slurry of plant protein and water with a cellulosic fiber in an amount from about 1% (wt. % of the slurry), or from about 3%, to about 10%, or to about 8%, or to about 7% wherein, optionally, the mixing of the cellulosic fiber with the slurry of plant protein, water uses a rotational mixer rotating at a rotation a speed at least about 2000 rpm, or at least about 2500 rpm at least about 3000 rpm or to at most about 5000 rpm; wherein, optionally, the fiber is selected from the group consisting of apple fiber, citrus fiber, pea fiber, potato fiber, wheat fiber, bamboo fiber, carrot fiber, psyllium and mixtures thereof.
96 The method of any one of claims 86 to 95 further comprising mixing a hydrocolloid with the slurry of plant protein and water wherein, optionally, the hydrocolloid is selected from the group consisting of agar, alginate, carragenan, methylcellulose, carboxymethylcellulose, hydroxypropylmethylcellulose, guar gum, cassia gum, locust bean gum, konjac gum, konjac mannan, pectin, tara gum, gellan, xanthan, various modified gelling starches, and mixtures.
97 The method of any one of claims 86 to 96 further comprising adding to the slurry of plant protein and water an oil in an amount greater than 0% (wt % of the slurry), or at least about 5%, or at least about 10%, or from about 5%, or from about 10%, or from about 15%, to about 30% or about 25% or to about 20% wherein the slurry of plant protein, oil and water is mixed to form an emulsion.
98 The method of any one of claims 86 to 97 wherein the plant protein is a plant protein isolate having a protein content from about 70% (wt. % protein content, dry basis), or about from 75%, or from 80% to about 95%, or to about 90.
99 The method of any one of claims 86 to 98 wherein the plant protein is not deamidated or hydrolyzed.
100 The method of any one of claims 86 to 99 wherein at least part of the plant protein is a structured plant protein wherein, optionally, the structured plant protein is obtained from a legume protein, wherein, optionally, the plant protein is selected from the group consisting of pea, fava bean, chickpea, lentil, mung bean, and soy and mixtures thereof.
101 The method of any one of claims 86 to 100 further comprising mixing a structured plant protein with a slurry of plant protein and water wherein, optionally, the structured plant protein is a legume protein, wherein, optionally, the plant protein is selected from the group consisting of pea, fava bean, chickpea, lentil, mung bean, and soy and mixtures thereof.
102 The method of any one of claims 86 to 101 further comprising placing at least part of the slurry comprising transglutaminase in a mold and allowing the slurry to incubate.
103 A gelled plant protein composition made by a process as described in any foregoing claim wherein, optionally, the composition is part of a vegan meat alternative, or vegetarian meat alternative, or meat containing composition wherein, optionally, the vegan meat alternative is in the form selected from the group consisting of a patty, a nugget, a finger a cased sausage, a fish fillet, and a poultry breast.
104 Use of a gelled plant protein composition by a process as described in any foregoing claim as a vegan meat alternative or vegetarian meat alternative, or meat containing composition wherein, optionally, the vegan meat alternative, vegetarian meat alternative, or meat containing composition is in the form selected from the group consisting of a patty, a nugget, a finger a cased sausage, a fish fillet, and a poultry breast.

The technology described in this specification can be further understood with reference to the following examples, which are provided for illustrative purposes and are not intended to further limit the full scope of the inventions described in this specification.

### EXAMPLE 1—EFFECT OF TRANSGLUTAMINASE DOSE OF GEL QUALITY

Effect of dose of transglutaminase enzyme on fixed amount of plant protein in water was evaluated for the ability of the systems to form gels. Based on screening exercises, legume proteins were found to be good at forming gels in aqueous systems using transglutaminase Specifically, plant protein solutions having 12% protein in water (wt. % of the solution) and about 0.30% transglutaminase (wt. % of the solution) were made using one of mung bean, fava bean, pea and chickpea canola, hemp, sunflower, yeast protein, and flax seed isolates (all having at least 60% protein (wt. % protein content)). It was seen that the aqueous legume protein mixtures (i.e. mung bean pea, and fava bean) generally made strong, elastic gels. It was further generally seen that aqueous mixtures using other plant proteins (canola, hemp, yeast, and sunflower) did not form gels or demonstrate increased in-solution viscosity.

From this screening further evaluations were made using pea protein isolates from various commercial sources all having from about 80% to about 85% protein content by weight, dry basis. To make gels using pea protein isolate, mixtures of pea protein isolate, water, and transglutaminase were made. In this example. gels were made as follows. Protein was mixed (12% wt. % of the mixture) with water having temperature of 20° C. until mixture is homogenous. The mixture was heated to 95° C. and then cooled to 45° C. prior to adding transglutaminase. Transglutaminase was mixed (0.30% wt. % of the mixture) with water and protein until the mixture is homogenized. The mixture was placed in molds, which sat for 45 minutes at 48° C. Molds were then heated in water bath (95° C.) for 10 minutes. The molds were then refrigerated (4° C.) for storage. In the evaluated mixtures, transglutaminase content was varied as follows. Transglutaminase ( ) was mixed with protein and water in an amount of 0.05%, 0.10%, 0.20%, 0.25%. 0.30%, 0.35%, 0.40%, (wt. % transglutaminase relative to the mixture).

Gel quality was evaluated qualitatively, and results were as follows. Gel firmness increased with increased concentration of transglutaminase up to 0.30% usage of transglutaminase (wt. % of the mixture). Using at least 0.35% transglutaminase (wt. % of the mixture) formed gels that were observed to release water indicating they were too firm. Additionally, legume flavor of the gel was found to decrease with increased concentration of transglutaminase.

### EXAMPLE 2—EFFECT OF PROTEIN DOSE ON GEL QUALITY

Effect of plant protein concentration on gel firmness was evaluated using pea protein isolate (about 85% protein (wt. % protein content, dry basis)) in water and fixed amount of transglutaminase. In this example, gels were made as follows. Protein was mixed (12% wt. % of the mixture) with water having temperature of 20° C. until mixture is homogenous. The mixture was heated to 95° C. and then cooled to 45° C. prior to adding transglutaminase. Transglutaminase was mixed (0.30% wt. % of the mixture) with water and protein until the mixture is homogenized. The mixture was placed in molds, which sat for 45 minutes at 48° C. Molds were then heated in water bath (95° C.) for 10 minutes to inactivate the transglutaminase. The molds were then refrigerated (4° C.) for storage. In the evaluated mixtures, transglutaminase content was varied as follows.

Mixture made for gels had protein content ranging up to 12% (wt. % of the mixture), and results were as follows. It was observed that at least 3% (wt. % of the mixture) protein content was needed form a gel. When protein content exceeded 3% (wt. % of the mixture), gel strength increased with increasing protein content. With protein content from 3% to 5% (wt. % of the mixture) gels were soft and had little elasticity. Using at least 8% protein (wt. % of the mixture) formed firm, elastic gels.

### EXAMPLE 3—EFFECT OF PROTEIN SOLUBILITY ON GEL QUALITY

Pea protein isolates of varying solubility are available commercially from various vendors. Applicants made three samples to evaluate gel strength using internally made pea protein isolates obtained using an isoelectric point separation process and measured to have variable solubility. Pea protein isolate sample I was measured to have protein content of about 80% (wt. % protein content, dry basis) and solubility in water at 95° C. of 50.5% (wt. % dissolved protein relative to total protein). Sample 2 was measured to have protein content of about 85% (wt. % protein content, dry basis) and solubility in water at 95° C. of 67.4% (wt. % dissolved protein relative to total protein). Sample 3 was measured to have protein content of about 80% (wt. % protein content, dry basis)) and solubility in water at 95° C. of 78.1% (wt. % dissolved protein relative to total protein).

In this example, gels were made as follows. Protein was mixed (12% wt. % of the mixture) with water having temperature of 50° C. until mixture was homogenous. Transglutaminase was mixed (0.3% (wt. % of the mixture)) with water and protein until the mixture was homogenized. The mixture was incubated for 3 hours at 48° C., enzyme was inactivated in hot water bath (95° C., for 10 minutes) and then the samples were transferred to refrigerator (4° C.).

Gel quality was evaluated qualitatively, and results were as follows. Mixtures using Sample 1 pea protein isolate did not gel. Mixtures using Sample 2 and Sample 3 gelled, and the mixture using Sample 3, formed firmer more elastic gels than the mixture using Sample 2. Among samples that did gel, it was further observed that gel firmness and elasticity increased when process parameters-like temperature, hydration time, and shear-used to make gels were adjusted to increase protein solubility. It was observed that gel firmness and elasticity were increased, and bitter and bean-like flavors were decreased when protein was mixed with water at 20° C. It was observed that gel firmness and elasticity were further increased when protein was mixed with water at 20° C. and let rest in water for 24 hours following homogenization but before transglutaminase was added.

Effect of protein solubility on gel strength was further evaluated using batches of a commercially available pea protein isolate having about 85% protein (wt. %, protein content, dry basis)), that were used past the use by date on the product labeling (called Sample 4 and Sample 5).

Gels were made as follows. Protein was mixed (12% wt. % of the mixture) with water have temperature of 50° C. until mixture is homogenous. Transglutaminase was mixed (0.3% (wt. % of the mixture)) with water and protein until the mixture is homogenized. The mixture sat for 3hours at 48° C., enzyme was then inactivated by heating the mixture in water bath (95° C., for 10 minutes). Samples were then refrigerated at 4° C. for storage. Sample 4 was measured to have protein content of about 85% (wt. %, protein content. dry basis) and solubility in water at 95° C. of 54.7% (wt. % dissolved protein relative to total protein), and was observed to form a soft, inelastic gel with strong bitter and bean-like flavors. Sample 5 was measured to have protein content of about 85% (wt. % protein content, dry basis) and solubility in water at 95° C. of 60.2% (wt. % dissolved protein relative to total protein), and was observed to form a soft, inelastic gel with strong bitter and bean-like flavors. Gels made using Sample 4 and Sample 5 are shown FIG. 1a (left two gels). A knife was lightly run across the top of the gels. As seen, the gels separate show lack of elasticity limited ability to support itself.

Given these results a further experiment was done. Sample 4 and Sample 5 were dissolved in water (95° C.) and mixed with high shear (3,000 rpm for 10 min). Transglutaminase was added after the samples cooled down to 45° C. The mixtures sat for 3 hours at 48° C. Transglutaminase was mixed (0.3% (wt. % of the mixture)) with water and protein until the mixture is homogenized. The mixture sat for 3 hours at 48° C., enzyme was then inactivated by heating the mixture in water bath (95° C.) for 10 minutes. Samples were then stored at 4° C. was then transferred to a refrigerator 4° C. for storage. Both samples formed firm, elastic gels with significantly reduced bitterness and legume notes. Gels made using Sample 4 as described in this paragraph are shown in FIG. 1b (right sample). A knife was lightly run across the top of the gel. As seen, the gel maintained its shape showing that compared to FIG. 1a it has higher elasticity and is better able to support itself.

### EXAMPLE 4—EFFECT OF SHEAR ON SOLUBILITY OF PROTEINS

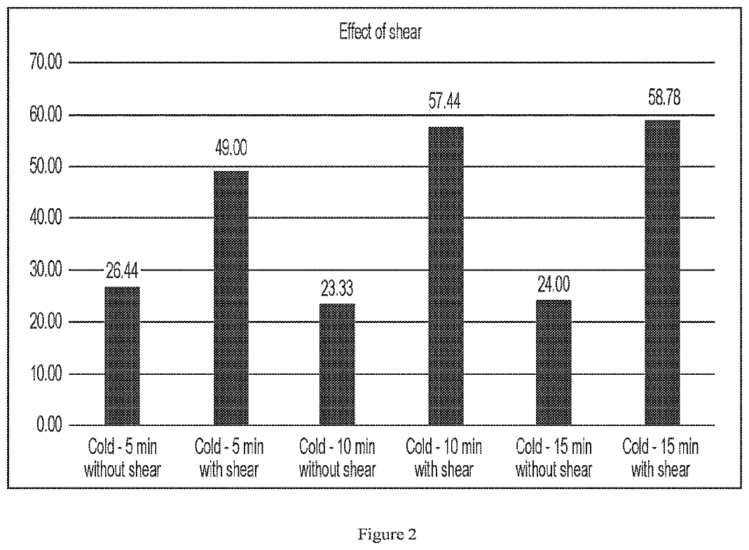

Alternate methods for increasing amount of protein dissolved in water were evaluated. In this example, pea protein isolate (about 85% protein (wt. % protein content, dry basis)) was mixed with water (3% protein in water (wt. % of the mixture)). Water had temperature of 20° C. Mixture was mixed using a high shear mixer that applies shear using a rotating mixing blade that was rotated at a rate of rate of 3000 rpm for 5, 10 or 15 minutes. Results are reported in FIG. 2, which reports a bar graph comparing the weight percent of pea protein dissolved compared to total protein in water after shear is applied for a defined time compared to the amount of protein that is dissolved in water after sitting for the same amount of time but without shear applied. With reference to FIG. 2, it is seen that applying shear for at least 5 minutes significantly increases the amount of protein dissolved in water compared to pea protein that is soaked in water for the same time without applied shear. It is further seen by applying shear by mixing pea protein in water at rotation rate of 3000 rpm for at least 10 minutes the dissolved protein in water content is greater than 57%. The amount of dissolved protein obtained by applying rotational shear as described for at least 10 minutes was found to be about like the amount of protein dissolved water at temperature of about 50° C.

In this example, following dissolving pea protein by applying rotation shear for 10 minutes, transglutaminase was added in an amount of 0.3% (wt. % of the mixture). The mixture of transglutaminase, protein and water was mixed (low speed and low shear) until homogenized. The mixtures sat for 3 hours at 48° C., was then heated in water bath (95° C.) for 10 minutes and was then transferred to a refrigerator 4° C. for storage. The gels obtained were self-standing, elastic and brittle.

### EXAMPLE 5—MODEL PROCESS FOR FORMING AN EDIBLE GELLED PLANT PROTEIN COMPOSITION

A gelled plant protein composition may comprise other components like fibers, hydrocolloids, oils, structured plant proteins, salts, and flavorings so that the composition is useful as a vegan or vegetarian meat alternative or meat extender composition. Cellulosic fibers, hydrocolloids, oils, structured vegetable proteins, salts, and flavorings are added to a gelled plant protein composition to adjust texture or taste of the composition to mimic a specific type of meat alternative. An illustrative process for making a gelled plant protein composition using cellulosic fibers, hydrocolloids, oils, structured vegetable proteins, salts, and flavorings is provided. The process may be adjusted to use one or more of a cellulosic fiber, a hydrocolloid, an oil, a structured vegetable protein, salts, and flavorings as needed.

Pour water into shearing mixer and add plant protein. A process is applied to increase the amount of protein dissolved to at least about 40% (wt. % of dissolved protein relative to plant flour, or plant protein concentrate, or plant protein isolate). The process may use one or more of high temperature, highly soluble protein isolate, or increased shear. Also, one more the foregoing variables can be used and the varied relative to each other to obtain the at least about 40% (wt. % of dissolved protein relative to plant flour, or plant protein concentrate, or plant protein isolate). For example in a process using a less highly water-soluble protein would need more shear to obtain the desired soluble protein content desired. In an illustrative method using shear, plant protein (at least about 3% (wt. % of the mixture)) is mixed in water (less than 50° C.) using shearing mixer rotating at a speed of at least about 2000 rpm, or at least about 2500 rpm, or at least about 3000 rpm so that at least about 40% (wt. % of dissolved protein relative to plant flour, or plant protein concentrate, or plant protein isolate) protein is dissolved in the slurry (at least about 5 minutes of shear applied). Once protein is dissolved other ingredients may be added to the slurry and are mixed until dispersed. Fibers may be added (about 1% to 10% (wt. % of the mixture)) and are dispersed using mixing speeds of at least about 2000 rpm. The shear disperses the fiber and may defibrillate or otherwise fragment the cellulosic fiber. Hydrocolloids may be added under lower shear (less than 1500 rpm) to disperse in the slurry. Oil may be added (about 5% to about 30% (wt. % of the mixture)) and the mixture may be mixed to form an oil-in emulsion by mixing at least at 2000 rpm. Structured plant protein may be added and can be hydrated before being added to the mixture of plant protein and water and other ingredients (mixing at less than about 1500 rpm) or more preferably is added after the emulsion is formed and all other shear steps are done so that the structured plant protein is not damaged by shear. Salt and flavorings can be added and dispersed using low shear (less than 1500 rpm). Ingredients can be added to water or to emulsion provided that adding and further mixing does not break the emulsion. Transglutaminase is added (from 0.001% to 0.35% of the mixture) to the slurry of protein and water and other ingredients or to the emulsion of oil, protein, and water and other ingredients. The mixture can be poured into molds to gel and take on the shape of a desired food product. Useful shapes include but are not limited to a patty, a nugget, a finger a cased sausage, a fish fillet, and a poultry breast. Favoring and texturizes can be chose to mimic any desired type of meat.

1. A method for obtaining a gelled plant protein composition comprising:
a. mixing a plant protein raw material with water having a temperature of less than about 50° C., to form a slurry having plant protein content of at least about 3% (wt. % of the slurry);
b. applying shear to the slurry so that at least about 40% (wt. % protein relative to the raw material) of the plant protein is dissolved in water,
c. adding transglutaminase to the slurry to form a mixture; and
d. incubating the mixture;
wherein the plant protein raw material is a legume protein; and
wherein the shear is applied by mixing the slurry of plant protein and water using a rotational mixer rotating at a rotational speed at least about 2000 rpm.

2. (canceled)

3. The method of claim 1 wherein the plant protein raw material is a plant protein isolate, a plant protein concentrate, a plant flour, or mixture thereof.

4. The method of claim 1 wherein the protein content in the slurry is in an amount from about 3% (wt. % of the slurry).

5. The method of claim 3 wherein the transglutaminase is mixed with the slurry in an amount from about 0.001% (wt. % of the slurry) to about 1.00%.

6. The method of claim 1 wherein the mixture is incubated for at least about 30 minutes, to about 24 hours.

7. The method of claim 1 wherein the mixture is incubated at a temperature greater than about 0° C., for a at least about 10 hours.

8. The method of claim 1 wherein the water has pH from about 5 to about 8.

9. (canceled)

10. (canceled)

11. (canceled)

12. The method of claim 1 wherein the shear is applied to the slurry for a time of at least about 5 minutes.

13. The method of claims 1 wherein the plant protein is selected from the group consisting of pea, fava bean, chickpea, lentil, mung bean, and soy mixtures thereof.

14. The method of claim 1 further comprising in step a) mixing a cellulosic fiber in an amount from about 1% (wt. % of the slurry)
wherein, optionally, the fiber is selected from the group consisting of apple fiber, citrus fiber, pea fiber, potato fiber, wheat fiber, bamboo fiber, carrot fiber, psyllium, and mixtures thereof.

15. The method of claim 1 further comprising in step a) mixing a hydrocolloid
wherein, optionally, the hydrocolloid is selected from the group consisting of agar, alginate, carragenan, methylcellulose, carboxymethylcellulose, hydroxypropylmethylcellulose, guar gum, cassia gum, locust bean gum, konjac gum, konjac mannan, pectin, tara gum, gellan, xanthan, various modified gelling starches, and mixtures thereof.

16. The method of claim 1 comprising mixing in step a) an oil in an amount of at least about 5%,
wherein the mixing forms an emulsion.

17. The method of claim 1 wherein the plant protein is a plant protein isolate having a protein content from about 70% (wt. % protein content, dry basis).

18. The method of claim 1 wherein the plant protein is not deamidated or hydrolyzed.

19. The method of claim 1 further comprising in step a) mixing a structured plant protein wherein optionally the structured plant protein is a legume protein, wherein optionally the structured plant protein is selected from the group consisting of pea, fava bean, chickpea, lentil and mixtures thereof.

20. The method of claim 1 further comprising placing at least part of the mixture comprising transglutaminase in a mold and allowing the mixture to incubate.

21-48. (canceled)

49. A gelled plant protein composition made by a process as described in claim 1
wherein, optionally, the composition is part of a vegan meat alternative, or vegetarian meat alternative, or meat containing composition
wherein, optionally, the vegan meat alternative is in the form selected from the group consisting of a patty, a nugget, a finger a cased sausage, a fish fillet, and a poultry breast.

50. Use of a gelled plant protein composition by a process as described in claim 1 as a vegan meat alternative or vegetarian meat alternative, or meat containing composition
wherein, optionally, the vegan meat alternative, vegetarian meat alternative, or meat containing composition is in the form selected from the group consisting of a patty, a nugget, a finger a cased sausage, a fish fillet, and a poultry breast.

51. The method of claim 2, wherein at least about 65% (wt. % dissolved protein relative to isolate, concentrate or flour) of the plant protein raw material dissolves in water at 95° C., centrifuged at 5000 rpm for 20 minutes.

52. The method of claim 1, wherein the transglutaminase is mixed with the slurry using a rotational mixer rotating at a rotation speed of from about 1000 rpm.

53. The method of claim 1, wherein the plant protein is a high in one or more of lysine and glutamine.
